(19) United States
(12) Patent Application Publication
(10) Pub. No.: US 2014/0228821 A1
(43) Pub. Date: Aug. 14, 2014

(54) DELIVERY CATHETER WITH CONTROLLED FLEXIBILITY

(71) Applicant: CONCEPTUS INC., Mountain View, CA (US)

(72) Inventors: Christopher A. Stout, San Bruno, CA (US); Julian Cruzada, San Jose, CA (US)

(73) Assignee: Conceptus Inc., Mountain View, CA (US)

(21) Appl. No.: 13/766,651

(22) Filed: Feb. 13, 2013

(51) Int. Cl. A61B 17/00

(52) U.S. Cl. CPC A61B 17/00
USPC 606/1

(57) ABSTRACT

A catheter in one embodiment includes an expandable implant and a delivery sheath having a lumen in which the expandable implant is disposed when the implant is being delivered. The delivery sheath includes a region having a plurality of openings designed to give a flexibility profile to the delivery sheath while allowing the implant to slide within the lumen, the outer surface of the implant being at least partially in contact with the openings during delivery of the implant.

### BACKGROUND OF THE INVENTION

Various approaches are known in the prior art to provide for a catheter that has different flexibilities at different regions of the catheter along the length of the catheter. In some uses of a catheter, the catheter may need to be relatively stiff at a proximal portion (in order to provide a sufficient level of pushability, which is the ability to transmit a force to the distal portion of the catheter from a proximal portion of the catheter) and may also need to be somewhat flexible at a distal portion (in order to provide a sufficient level of trackability, which is the ability of the distal portion to navigate pathways in a patient's body). One approach in the prior art uses a set of one or more wires or coils or braids that are embedded within a catheter's walls to reinforce a region or portion, such as a proximal portion of the catheter, while another portion does not include such reinforcement. The reinforcement can provide improved pushability in the proximal portion and a distal portion can have no reinforcement. However, this approach tends to increase the size, such as a cross-sectional diameter or area, of the catheter due to the added volume of the reinforcement. Another approach that is known in the art uses a spiral cut in the catheter's walls; examples of spiral cut catheters are described in U.S. Pat. No. 7,744,586 and U.S. Patent Application Publication No. 2009/0157048.

### SUMMARY OF THE DESCRIPTION

A catheter, in one embodiment of the invention, can include a medical device, such as an expandable implant having an outer surface, and a handle coupled to the expandable implant and a delivery sheath coupled to the handle. The delivery sheath has an outer surface and a lumen in which the expandable implant is disposed, and the lumen is defined by an inner surface of the delivery sheath. The delivery sheath includes a plurality of openings near a distal end of the delivery sheath, and the openings extend from the inner surface to the outer surface of the delivery sheath. In one embodiment, the openings are in direct physical contact with the outer surface of the expandable implant or other medical device, and the openings are configured in at least one of size, shape and orientation to allow the outer surface of the expandable implant to slide along the openings without having the outer surface catch or snag on one of the openings as the outer surface slides along the openings. In one embodiment, the expandable implant can include a first coil which forms the outer surface of the expandable implant and a second coil that is coupled to the first coil and is coaxially surrounded by the first coil; the expandable implant can also include an expandable hydrogel that swells once the implant is deployed in a physiological environment (e.g., deployed within a fallopian tube). In one embodiment, the distal end of the delivery sheath is sized to allow the expandable implant to be deployed through the distal end and the expandable implant slides along the openings as the expandable implant is deployed. In one embodiment, the plurality of openings are configured to make the delivery sheath more flexible in the region containing the plurality of openings than a region of the delivery sheath which does not contain the openings.

The openings, in one embodiment, can have a shape selected from: a triangle, a four or more sided polygon such as a quadrilateral, or a closed form curve (such as a circle, an oval, an ellipse, etc.). In one embodiment, the openings can vary in size such that openings near the distal end are larger than openings that are proximal of the distal end; this variation can occur across zones in which the openings within a zone are the same size and the openings, from zone to zone, become progressively larger towards the distal end of the delivery sheath. In one embodiment, the sheath can have different regions or zones of openings in which the density of openings differs between the regions or zones; for example, for a sheath having two regions (a first and a second), the first region can have a higher density (in terms of surface area) of openings than the second region. In one embodiment, the openings in these different density regions can have the same size (e.g., they are each squares or shapes having sides less than 0.05 inches in length); in another embodiment, the openings in the denser region (e.g. the first region which is distal of the second region) are larger than the openings in the less dense region. In one embodiment, the openings can be disposed in a portion on a distal end of the delivery sheath that is about 0.5 inches to about 6 inches long; this portion can terminate at the open distal end of the delivery sheath and extend proximally 0.5 inches to 6 inches from the open distal end. In one embodiment, the orientation of each of the openings is configured to minimize any resistance or friction that can occur as the expandable implant slides along the openings; for example, in one embodiment, no side of each of the openings is perpendicular to a longitudinal axis of the delivery sheath, wherein the longitudinal axis extends down the length of the sheath from the distal end of the proximal end of the sheath. This orientation reduces in one embodiment the size of any edges that might be flush with an advancing edge of the implant as it slides along the openings.

One embodiment can include a sleeve disposed over the outer surface of the delivery sheath in one or more regions containing the openings. This sleeve can at least partially restrict the flow of fluid through the openings into the lumen of the delivery sheath; for example, if the expandable implant includes a swellable hydrogel component, the sleeve can restrict the flow of fluids into the lumen to reduce any swelling of the hydrogel while the hydrogel is in the lumen. In one embodiment, the sleeve can be formed from a composition that is different than the composition forming the delivery sheath such that the sleeve is much more flexible than at least the proximal portion of the sheath and can be as flexible as or more flexible than the distal portion of the sheath which includes the plurality of openings. Such a sleeve can retain the flexibility and/or trackability of the distal portion of the delivery sheath while restricting the flow of fluids into the lumen of the delivery sheath. In one embodiment, the distal open end of the delivery sheath can also include a material that is place on or into the distal open end in order to restrict the flow of fluid into the distal open end; for example, the material can be a gel or jelly that is stuffed into the distal open end. In one embodiment, the material can be one of: (a) a pierceable hydrophobic or hydrophilic material or (b) a pierceable seal or cap that attaches to the distal end or (c) a dissolvable seal or cap that attaches to the distal end. In one embodiment, a distal end of the implant can extend out beyond the distal open end of the delivery sheath while the material at least partially restricts the flow of fluid into the distal end.

In one embodiment, the delivery sheath can include a solid band or section, along the longitudinal length of the delivery sheath, that interrupts or separates one set of openings from another set of openings. The solid band can be positioned, along the longitudinal length, at a predetermined point that tends to kind, if the solid band is not present, when the delivery sheath is used, in a typical physiological setting, to deploy the expandable implant. The solid band is positioned to resist the kinking which tends to occur when the solid band is not present. In one embodiment, the solid band is positioned at a point that is about 10 to about 25 mm from the distal end of the delivery sheath.

A delivery sheath, according to one embodiment, can have a constant or consistent wall thickness through the entire length (from proximal end to distal open end) of the delivery sheath and yet still have a variation in flexibility to provide sufficient pushability at the proximal end (which has no openings), and the openings at the distal end can provide sufficient flexibility to provide trackability. Moreover, this wall thickness can be less than the thickness of the wall of a catheter reinforced with coils or braids. Hence, according to this embodiment, the delivery sheath can have a smaller cross section than such a reinforced catheter while still providing a variation in flexibility. The openings can be configured in at least one of size, shape and orientation to allow an outer surface of a medical device, in a lumen of the sheath, to slide along the openings. In one embodiment, each of the openings can be separate and distinct from the other openings and each can have a non-negligible surface area. The openings can be dispersed evenly around the circumference of a cross-section that is perpendicular to the longitudinal axis of the delivery sheath.

A delivery sheath, according to one embodiment, can have a variation in diameter or in wall thickness from proximal to distal ends and also include openings designed to provide a variation in flexibility. For example, the wall thickness of a sheath, in one embodiment, can change from a first thickness near a proximal end to a second, smaller thickness near a distal end; also, the sheath diameter can change, such as a larger diameter near a proximal end to a smaller diameter near a distal end such that the sheath has a tapered profile.

A delivery sheath, according to one embodiment, can have dimples instead of openings or in addition to openings. The dimples can be depressions in one or more surfaces (inner and/or outer surfaces) of the delivery sheath, and the dimples can provide a variation in flexibility in the portion of the sheath that includes the dimples relative to other portions of the sheath that do not include the dimples. The portion can be a distal portion of the sheath. The dimples, in one embodiment, are separate and distinct from each other and can have a shape selected from one of: (a) a closed form curve such as a circle, oval or ellipse; (b) a triangle; or (c) a polygon having four or more sides. The dimples can be arranged in patterns of dimples such as different regions of dimples having different sizes or different densities or different sizes and different densities, etc.

The foregoing summary is not intended to be a complete summary of the detailed description, which follows.

The above summary does not include an exhaustive list of all aspects of the present invention. It is contemplated that the invention includes all systems and methods that can be practiced from all suitable combinations of the various aspects summarized above, and also those disclosed in the Detailed Description below.

### BRIEF DESCRIPTION OF THE DRAWINGS

The present invention is illustrated by way of example and not limitation in the figures of the accompanying drawings in which like references indicate similar elements.

FIG. 1A shows a side view of an embodiment of an expandable implant device which can be used with a delivery sheath described herein.

FIG. 1B shows a side view of the implant device of FIG. 1A within a delivery sheath according to one embodiment; the delivery sheath 25 is shown in a cut away view without any openings.

FIG. 1C is a side view showing an alternative embodiment of an expandable implant device.

FIG. 2A is a side view of a delivery sheath according to an embodiment of the invention.

FIG. 2B is a side view of a delivery sheath according to an embodiment of the invention.

FIG. 2C is a side view of an embodiment of a delivery sheath having a plurality of zones of openings.

FIG. 2D is a side view of an embodiment of a delivery sheath having a plurality of zones of openings.

FIGS. 3A, 3C, and 3E are side views of an embodiment of a delivery sheath.

FIG. 3B is a detailed side view of a portion of a distal zone (zone 1) shown in FIG. 3A.

FIG. 3D is a detailed side view of a portion of another zone (“C”) shown in FIGS. 3C and 3A.

FIG. 3F is a detailed side view of a portion of another zone (“D”) shown in FIG. 3E.

FIG. 4A shows a side view of a portion of a delivery sheath of one embodiment.

FIG. 4B shows a detailed view of one of the openings on the delivery sheath shown in FIG. 4A.

FIGS. 4C, 4D, and 4E show detailed views of alternative embodiments of openings for one or more delivery sheaths.

FIG. 5A shows a cross-sectional view of delivery sheath 301 in FIG. 3A, the cross-section taken at line FIG. 5 as shown in FIG. 3A.

FIG. 5B is a cross-sectional view of the delivery sheath 301 with the implant device 10 disposed in the lumen of sheath 301 for deployment of the implant device.

FIG. 6 is a side view of an embodiment of a delivery sheath which includes a solid band designed to resist kinking at an expected possible kink point.

FIG. 7A is a side view of an embodiment of a delivery sheath which includes a sleeve covering at least some of the openings (shown by dashed lines) on the delivery sheath.

FIG. 7B is a cross-sectional view of the sheath shown in FIG. 7A, the cross-sectional view taken at line 7B-7B shown in FIG. 7A.

FIG. 8A is a side view of an embodiment of a delivery sheath which includes a sleeve and material disposed in the distal open end of the sheath.

FIG. 8B is a side view of the embodiment shown in FIG. 8A after a distal portion of an implant device has been deployed beyond the distal open end of the sheath and beyond the material disposed in the distal open end.

FIG. 9A is a side view of an embodiment of a delivery sheath which includes dimples in the outer surface of the sheath.

FIG. 9B is a cross-sectional view of the sheath shown in FIG. 9A, the cross-sectional view taken at line 9B-9B shown in FIG. 9A.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment. Although processes are described below in terms of some sequential operations, it should be appreciated that some of the operations described may be performed in a different order. Moreover, some operations may be performed in parallel rather than sequentially.

This disclosure provides various embodiments of a catheter system that can have different flexibilities, or other behavioral characteristics, at different regions of the system. The various embodiments described herein can be used with many different types of medical devices even though this disclosure focuses on expandable implants for fallopian tube occlusion. Thus, it will be understood that one or more embodiments described herein can be used to deliver one or more stents (such as stents for coronary artery angioplasty or other types of stents) or to deliver one or more devices to treat aneurysms or to deliver one or more devices to perform diagnostic operations or to deliver one or more devices to occlude the vas deferens or to deliver one or more devices to perform other medical operations (such as delivering a drug to a location in a patient's anatomy) etc. It will also be understood that the embodiments of the sheath described herein can be used in various different ways as part of a catheter system; the sheath could be on the exterior of the system or could be within the lumen of a tubular structure or cannula. The sheath could alternatively include multiple lumens which can each have a set of openings as described herein.

In one embodiment, a delivery sheath as described herein can be used to deliver a fallopian tube expandable implant device that will occlude the fallopian either immediately or a few weeks after the delivery of the implanted device. The implant device can self-expand once it is deployed from the delivery sheath, and the deployment of the implant device can be controlled from a handle that is coupled to the implant device and is coupled to the delivery sheath at the proximal end of the delivery sheath. The implant device can be deployed transcervically through the delivery sheath that can be introduced, in one embodiment, through the cervix with a hysteroscope which allows a medical practitioner to locate the ostium of each fallopian tube in order to track the delivery sheath into the fallopian tube. The handle, in one embodiment, can be used to push the delivery sheath into the fallopian tube and to then deploy the implant device into the fallopian tube. In one embodiment the handle can be used to retract the delivery sheath, once it is properly positioned within the fallopian tube, to expose the implant device to the walls of the fallopian tube, and the implant device can be released, through a control on the handle, so the implant device can expand and engage the walls of the fallopian tube. In one embodiment, the implant device can be similar to the Essure device from Conceptus, Inc. of Mountain View, Calif. Further information about the procedures involved in deploying such devices and the handles used in controlling the deployment of such devices is provided in U.S. Pat. No. 7,506,650 and U.S. Patent Application Publication Nos. 2008/0041394 and 2011/0094519 and each of these three patent documents are incorporated herein by reference in their entirety.

The expandable implant may be formed from metal such as stainless steel or a superelastic or shape memory material such as a nickel titanium (NiTi) alloy such as nitinol, or platinum, or tantalum, or gold, or rigid or semi-rigid biocompatible plastics. In one particular embodiment, the expandable implant may be formed at least in part from a superelastic material providing a controlled force on the body lumen such as a portion of the fallopian tube during expansion of the implant. The implant may self-expand radially from a first diameter to a second diameter which is larger than the first diameter. The implant may be delivered by a delivery system (e.g. a delivery catheter which includes a delivery sheath as described herein) which constrains the implant to the size of the first diameter and after the implant is deployed, it may expand to the second diameter which at least slightly exceeds the diameter of a lumen of the fallopian tube. The material or materials of the implant may be superelastic so that the implant can expand in a manner that causes it to resiliently apply an anchoring force against the wall of the fallopian tube, thereby resisting against being expelled by the fallopian tube.

The surface of the implant may be designed to facilitate epithelial growth; one way of doing this is to provide the implant with an open or latticelike framework to promote and support epithelial growth into as well as around the implant to ensure secure attachment to the implant within the wall of the body lumen. The implant may include a tissue ingrowth promoting agent such as a polyester fiber (e.g. polyethylene terephthalate) or other materials known to facilitate fibrotic or epithelial growth. The surface of the implant may also be modified or treated or include such a tissue ingrowth promoting material. The surface modification may include plasma deposition or laser drilling or photochemical etching or sintering and the like. Further, increasing the surface area of the implant by such surface modification techniques (e.g. surface drilling or etching or sintering) can also provide greater adhesion for the epithelial tissue. Suitable surface treatments include plasma etching, sandblasting, machining and other treatments to roughen the surface. In other embodiments, the implant may be coated or seeded to spur epithelialization. For example, the implant can be coated with a polymer having impregnated therein a drug, enzyme or protein for inducing or promoting epithelial tissue growth. Any of these various techniques for including a tissue ingrowth promoting agent may be used with the various other implants shown or described herein. The implant can, in one embodiment, also include one or more hydrogel components that can swell by absorbing fluid once the implant is deployed, and further details regarding such hydrogel components are described in U.S. Application Publication No. 2011/0094519.

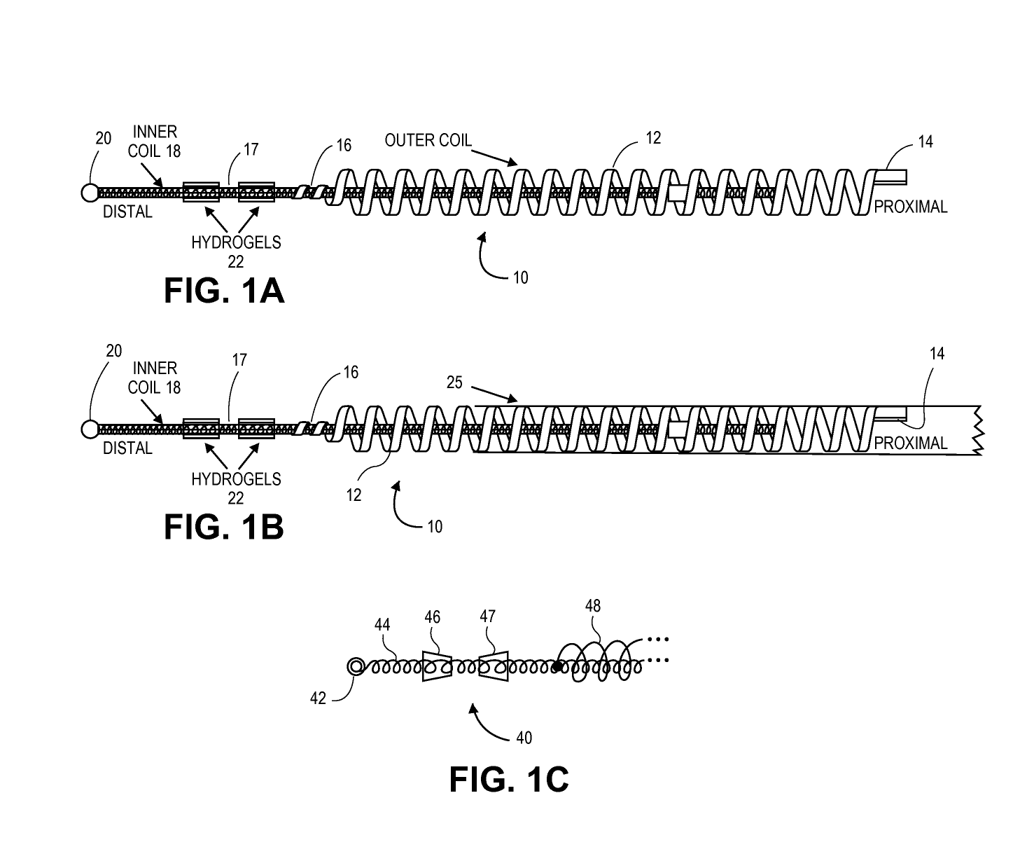

FIG. 1A shows an embodiment of a fallopian tube implant device 10 (after it has been detached from a delivery mechanism such as a core wire that fits into fitting 14). The implant device 10 can include an outer coil 12 that is coupled to an inner coil 18 at a joint 16. The outer coil 12 and the inner coil 18 can be similar to the outer and inner coils on the Essure device from Conceptus, Inc. The joint 16 can be any one of a set of possible couplings, such as a solder joint or glue or a mechanical crimping of the two coils or an strap or bundle of fiber, etc. The outer coil 12 can be coaxially surround inner coil 18 which can coaxially surround an optional flexible rod or wire 17 (shown between the hydrogel components 22 at a stretched portion of inner coil 18). The inner coil 18 can be stretched near the hydrogel components 22, including the portion of inner coil 18 between the hydrogel components 22 and the portion of inner coil 18 covered by the hydrogel components. While FIG. 1A shows two such hydrogel components, alternative embodiments can use fewer or more such hydrogel components. Further information about hydrogel components is provided in U.S. Patent Application Publication No. 2011/0094519 which is hereby incorporated herein by reference. In another embodiment, one or more hydrogel components can be disposed on the implant device at locations other than near the distal end of the inner coil 18; for example, the one or more hydrogel components can be disposed on a proximal portion of inner coil 18 or be disposed on all, or a portion, of outer coil 12 or be disposed on the distal ball 20 which can be an atraumatic ball that is coupled to at least one of the inner coil 18 and the flexible rod or wire 17 (shown between the hydrogel components 22). While tissue ingrowth promoting agents, such as Dacron or polyester fibers or other agents described herein, are not shown in the implant devices in FIGS. 1A-1C, it will be understood that these implant devices can include such tissue ingrowth promoting agents. The implant device 10 can be coupled to a handle (not shown) such as the handles described in U.S. Pat. No. 7,506,650, incorporated herein by reference, and U.S. Patent Application Publication No. 2008/0041394, incorporated herein by reference. The handle can be used to guide the insertion of the implant into a fallopian tube and to deploy the implant within the fallopian tube. The handle can be coupled to the implant device 10 by a corewire (not shown in FIGS. 1A, 1B, and 1C) that is coupled, at the corewire's proximal end, to a mechanism in the handle that can pull the distal end of the corewire out of fitting 14 which can be an interference or friction fitting that couples the outer coil 12 to the distal end of the corewire. In one embodiment, the distal end of the corewire can fit inside of one end (the proximal end) of fitting 14 and the inner coil 18 can fit within the other end (the distal end) of fitting 14 which can be shaped like a tube. In one embodiment, before deployment, the corewire, outer coil 12 and inner coil 18 are coupled together at the fitting 14 and are disposed within the lumen of a delivery sheath, as any one of the delivery sheaths described or shown herein, such as delivery sheaths 25, 201, 211, 221, 231, 301, 401, 601, 701 or 801. In one embodiment, the outer coil 12 will be restrained, prior to being deployed, against the inner wall of the delivery sheath such that the outer coil 12 exerts an outward force against the inner wall of the delivery sheath and hence contacts and abuts and slides against that inner wall when the outer coil 12 and the inner wall move relative to each other (such as when the delivery sheath in one embodiment is retracted proximally toward the handle in response to the activation of the handle's release mechanism which can pull back (proximally) the delivery sheath toward the handle). In one embodiment, when the corewire is pulled proximally toward the handle, the corewire is released from its coupling with the fitting 14 and the inner coil 18 is released from its coupling with fitting 14, resulting in the configuration of implant device 10 shown in FIGS. 1A and 1B and 1C. In one embodiment, the delivery sheath, such as sheaths 25, 201 or 301, can be retracted proximally toward the handle, before the corewire is pulled proximally toward the handle, so that the outer coil 12 is partially or fully exposed (outside of the delivery sheath) before the corewire is pulled proximally toward the handle; in another embodiment, the delivery sheath can be retracted after the corewire is detached from the proximal end of fitting 14 (by pulling the corewire proximally toward the handle).

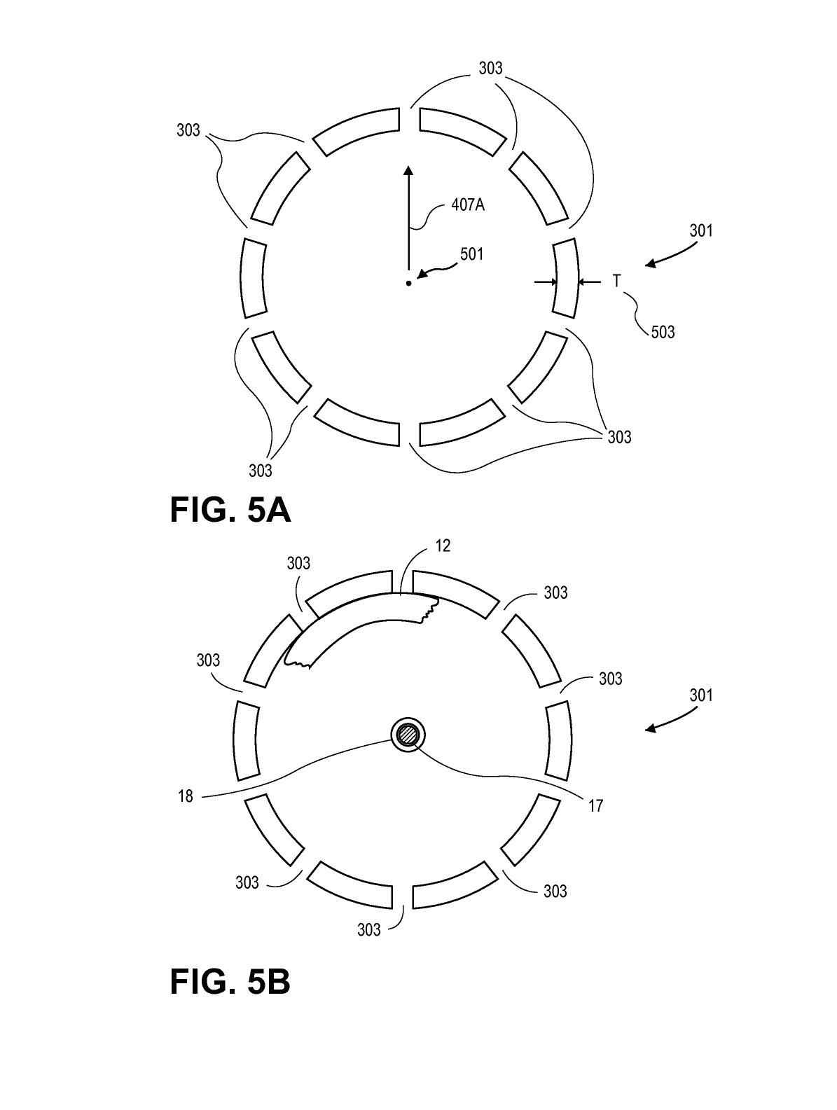

FIG. 1B shows the implant device 10 as it is being deployed in one embodiment. In the embodiment shown in FIG. 1B, the corewire has been decoupled from fitting 14 before (or as) the delivery sheath is fully retracted proximally to expose the outer coil of the implant device 10. In another embodiment, the corewire can still be attached to the fitting 14 as the delivery sheath 25 is being retracted proximally toward the handle, such that the outer coil can be partially or fully exposed outside of the delivery sheath 25 before the corewire is decoupled from the fitting 14. FIG. 1B shows how the outer surface of the outer coil contacts and slides against the inner surface of the delivery sheath 25 which includes openings such as the openings described herein (e.g., openings 207A, 214A, 224, 234, 303, etc.). Thus, when delivery sheath 25 is moved relative to implant device 10 in order to deploy the device 10, the outer surface of outer coil 12 slides against the openings (also see FIG. 5B); improperly sized or shaped or oriented openings can snag an edge of the outer coil 12 and this snagging can lead to a failed deployment or to a failed device. The openings in delivery sheath 25 are not shown in the partial cut out view of sheath 25, but it will be understood that delivery sheath 25 can have any one of the various embodiments of openings described herein (e.g. openings 207A or 224 or 303 or 405 or 410 or 433 or 437, etc.).

FIG. 1C shows an alternative embodiment of an implant device which is implant device 40. The implant device 40 is similar to implant device 10 except it includes two truncated cone-shaped hydrogels 46 and 47 that can be formed separately and then glued together, with their smaller ends facing each other, and are glued together at the stretched out portion of inner coil 44. The implant device 40 also includes an outer coil 48 that is coupled to the inner coil 44 at a joint, and the inner coil 44 is coupled to an atraumatic distal ball 42.

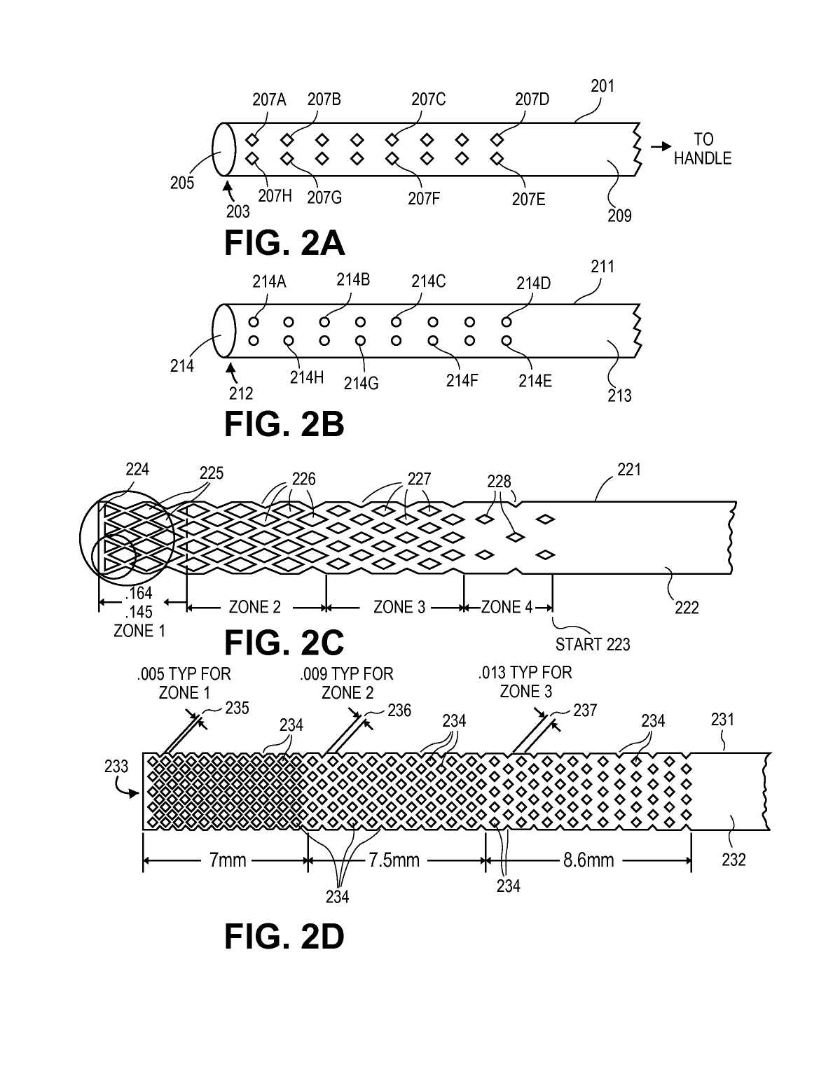

A variety of different openings and different configurations and patterns of openings for various delivery sheaths will now be described while referring to FIGS. 2A, 2B, 2C, 2D, 3A, 3B, 3C, 3D, 3E, 3F, 4A, 4B, 4C, 4D, 4E, 5A, 5B, 6, 7A, 7B, 8A, 8B, 9A, and 9B. These various delivery sheaths can be used to deliver the expandable implant devices shown in FIGS. 1A-1C or other types of devices or to perform other operations; these various delivery sheaths can, for example, be used as the sheath 14 in U.S. Pat. No. 7,506,650 to deliver the device 12 in U.S. Pat. No. 7,506,650. It will be appreciated that FIGS. 2C, 2D, 3A, 3C, 3E, and 6 show representations of a flattened pattern of openings as if the sheath or tube was cut longitudinally and flattened out.

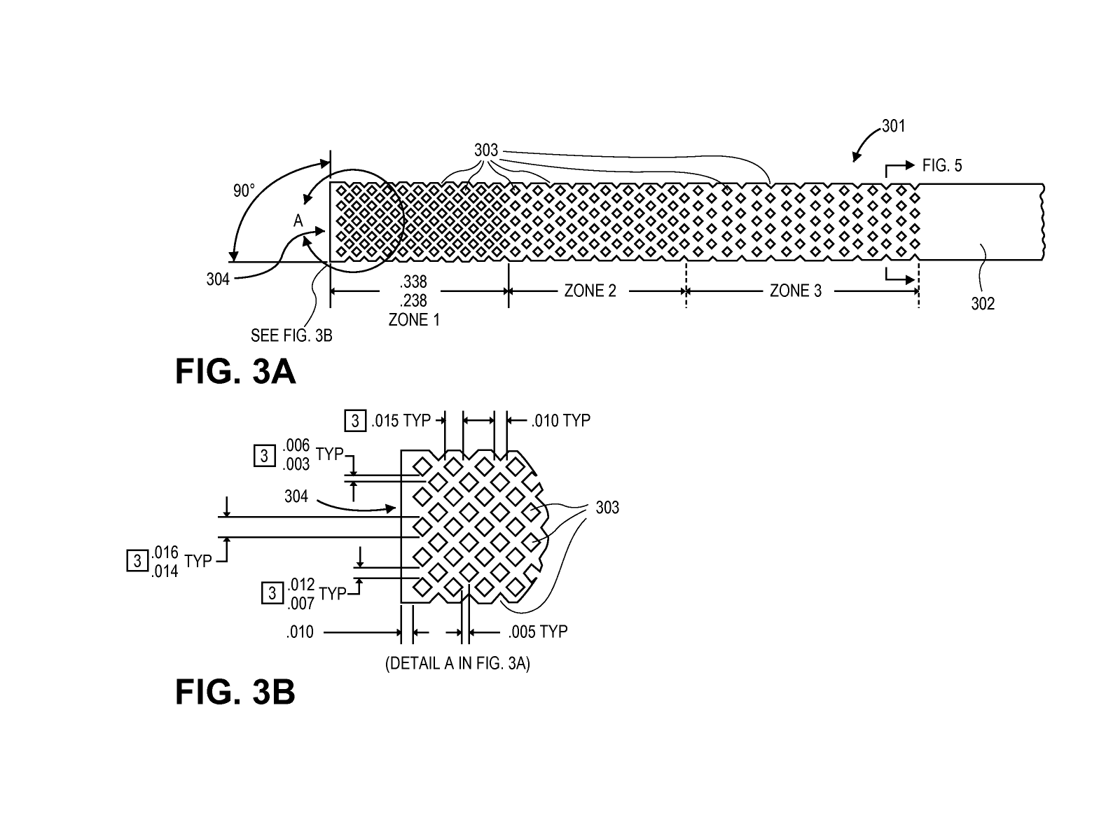

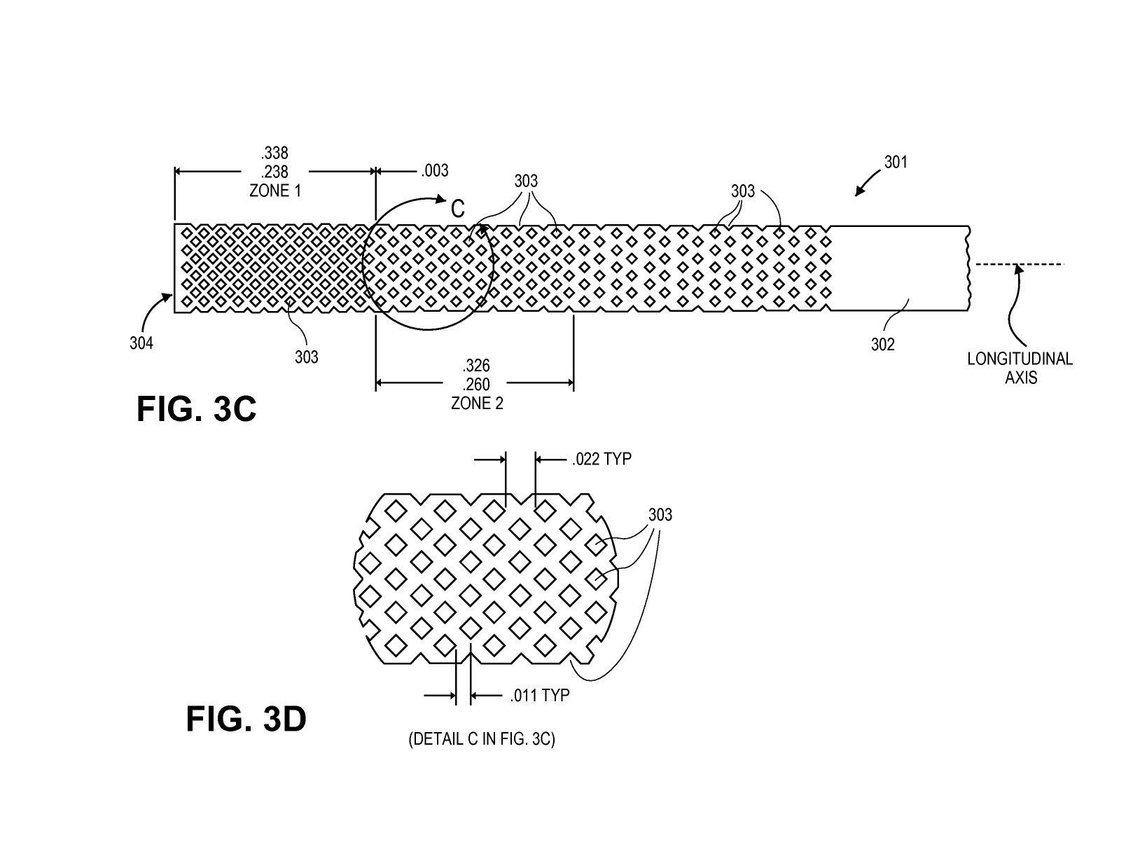

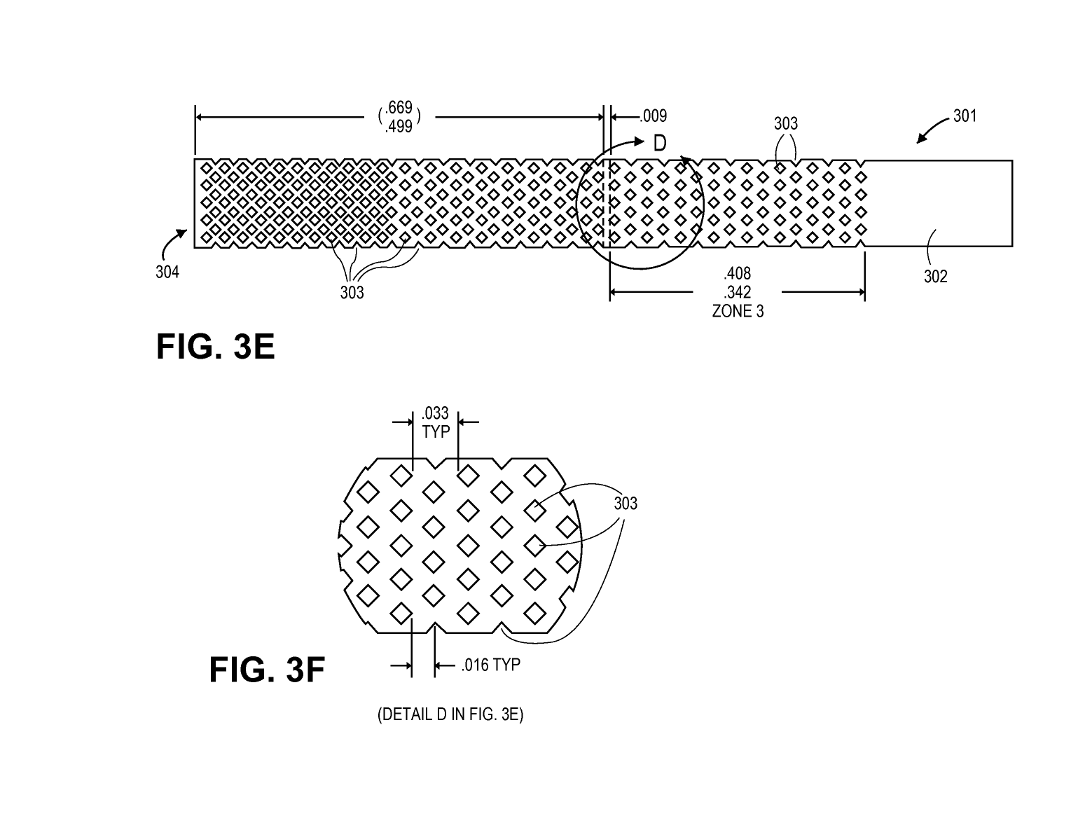

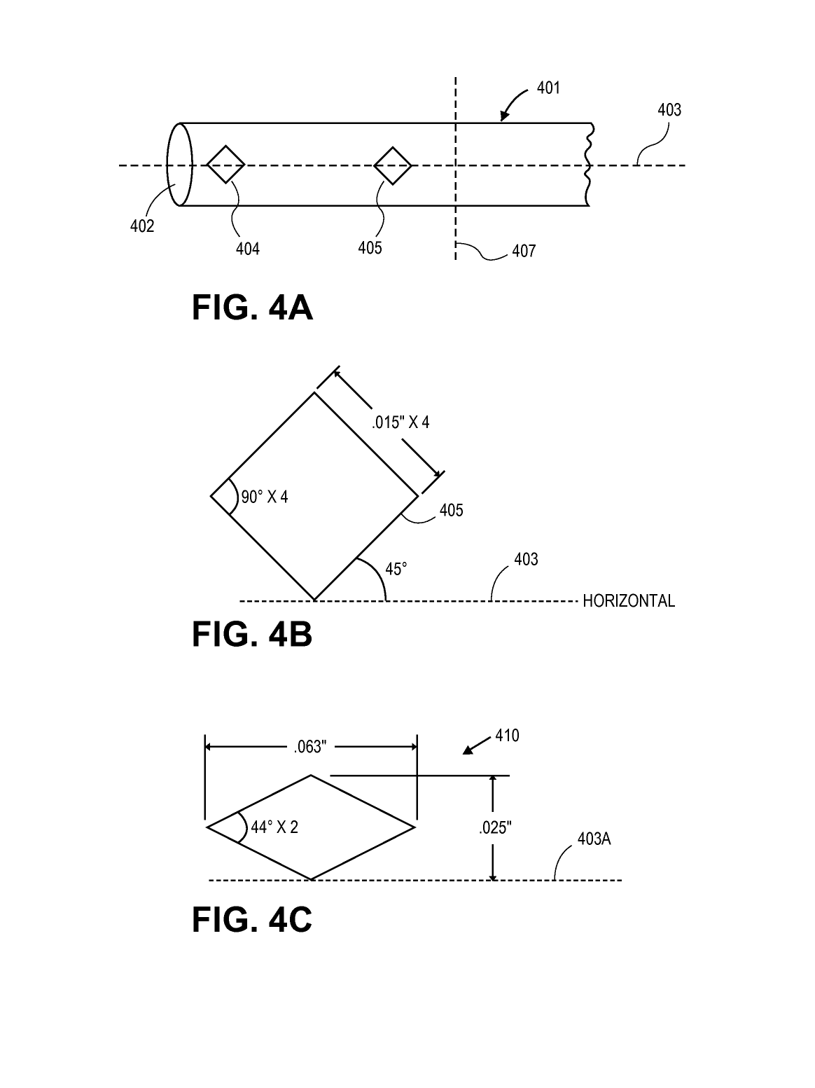

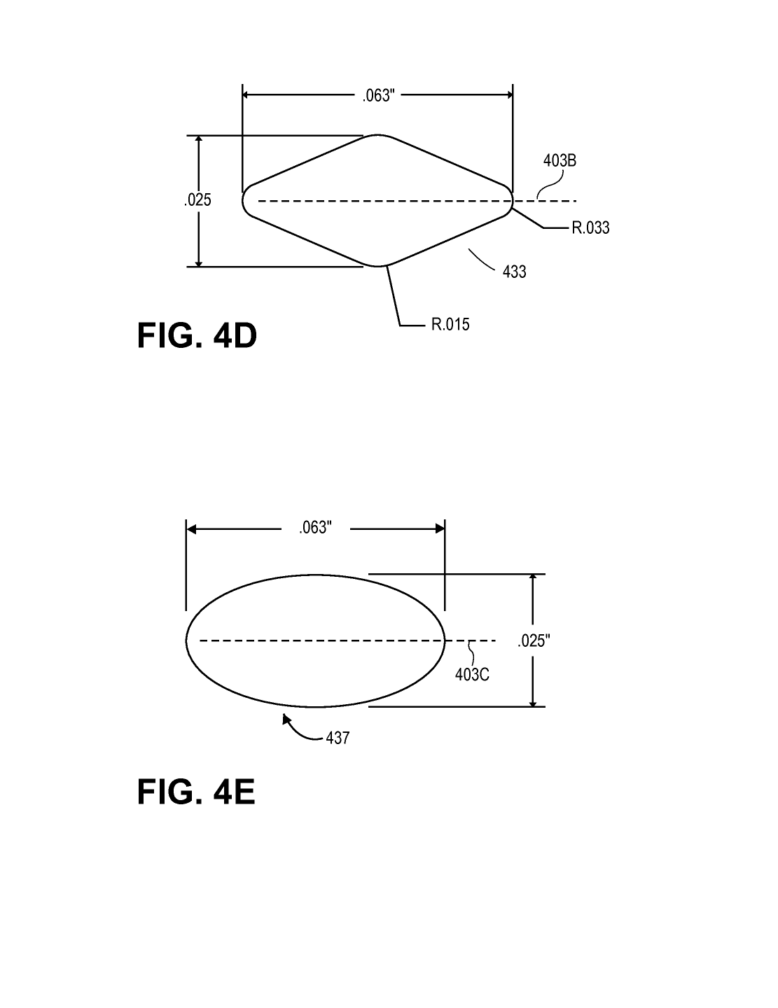

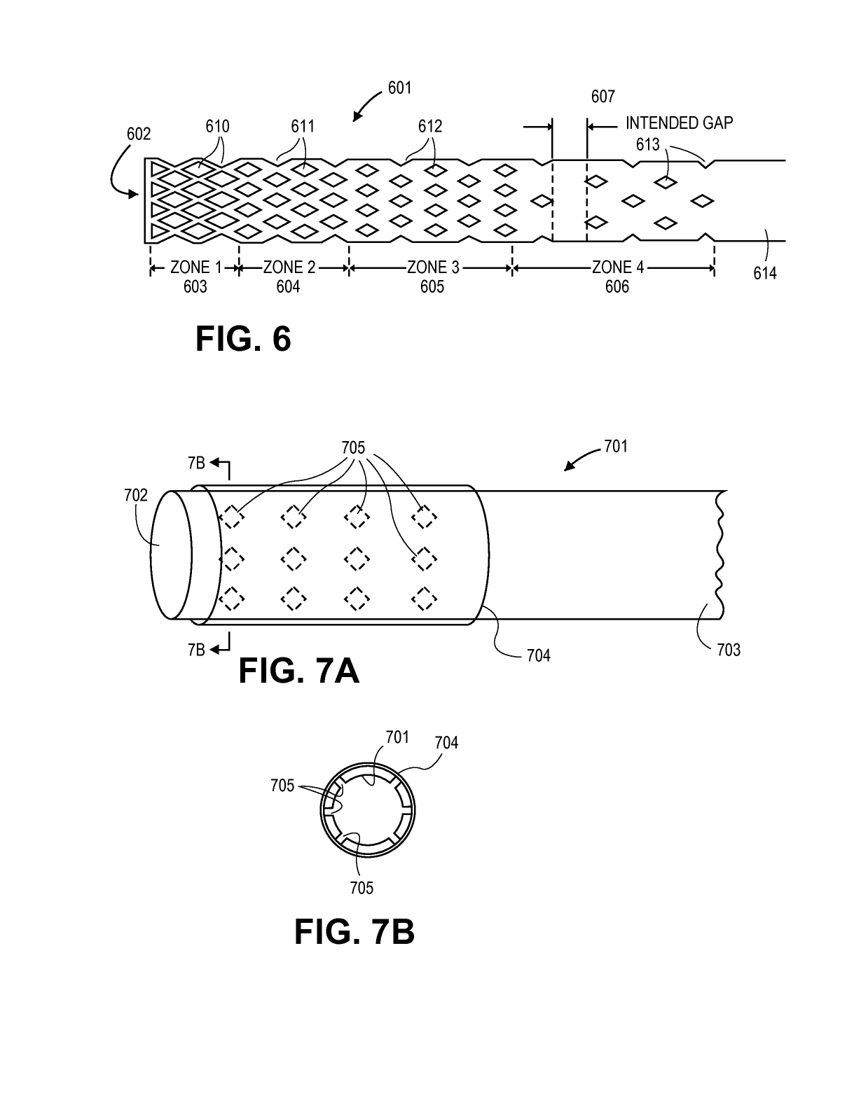

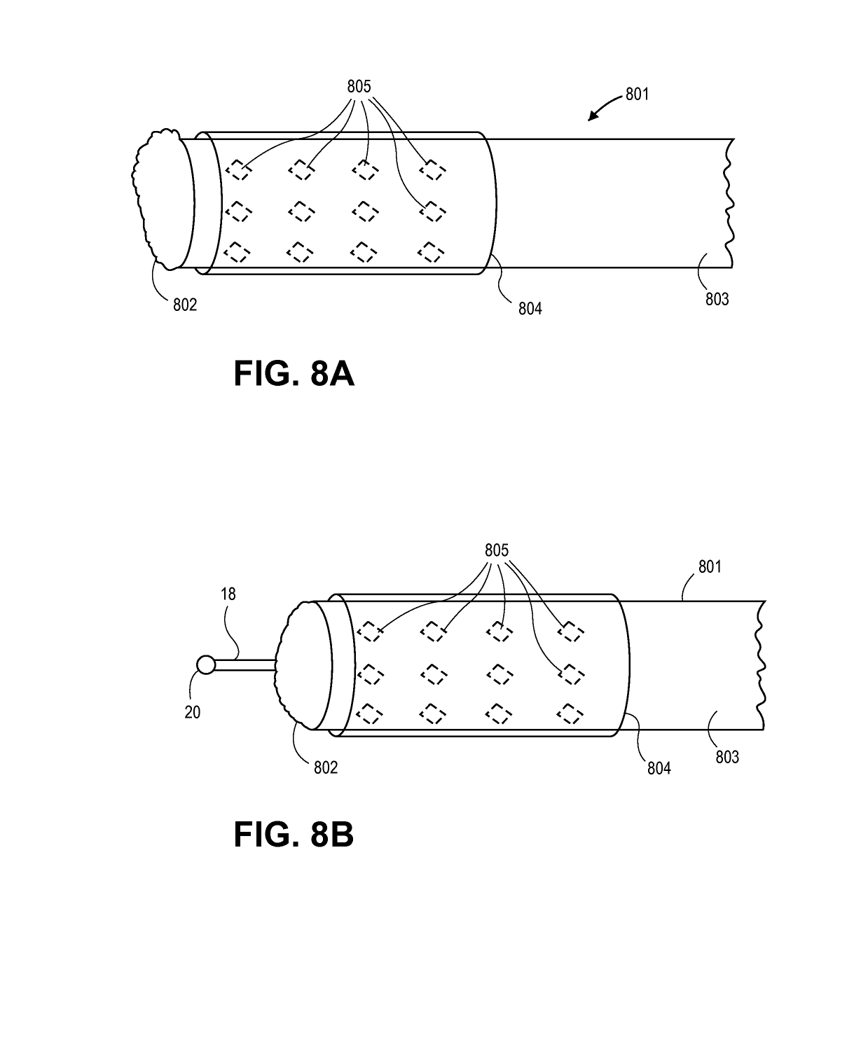

FIG. 2A shows an example of a delivery sheath according to one embodiment. Delivery sheath 201 shown in FIG. 2A includes a distal end 203 and a distal open end 205 which is an opening into the lumen of the delivery sheath 201. The proximal end of delivery sheath 201 can, in one embodiment, be coupled to a handle such as the handle described in U.S. Pat. No. 7,506,650, which is incorporated herein by reference. A plurality of openings are shown on the delivery sheath 201, including openings 207A through 207H. In the embodiment shown in FIG. 2A, each of the openings has the same size, and there is a constant density of the openings across the area occupied by the openings. There is no overlap between the openings (unlike the embodiments shown in FIGS. 2C and 2D) and there is no solid band or intended gap (such as in the embodiment shown in FIG. 6). Each of the openings, such as openings 207A through 207H can be a square or quadrilateral. The openings can be of a small surface area as described further below, such as in relation to FIGS. 3A through 3F or FIGS. 4B through 4E. Each of the openings can be configured in at least one of size, shape and orientation to allow the outer surface of the device being deployed through the lumen of delivery sheath 201 to slide along the openings. For example, each of the openings can have a small size and an appropriate shape as described herein and an orientation such that outer coil 12 of the implant shown in FIG. 1A can slide along the inner surface of the openings without snagging on those openings. Each of the openings is discrete and separate from the other openings (unlike a spiral cut on a catheter) and each can have a non-negligible surface area. In one embodiment, the orientation of each of the openings is set such that no edge of the openings is perpendicular to a longitudinal access of the delivery sheath which extends from the proximal to the distal end of the delivery sheath (for example see FIG. 4A). In one embodiment, the angles formed by each pair of sides can be bisected by the longitudinal axis as shown in FIG. 4A, and this orientation tends to reduce the snagging of outer coil 12 as the implant is moved relative to the delivery sheath, such as when the delivery sheath is retracted proximally towards the handle. In one embodiment, only a small portion of a distal part of the delivery sheath contains or includes the openings; for example, the last inch or six inches of a delivery sheath may include the openings while the remainder of the delivery sheath, which can be over 40 centimeters long will not include such openings. As shown in FIG. 2A, the openings begin near the distal end 203 and progress proximally up until portion 209 which is proximal of the distal end. In one embodiment, the openings, such as openings 207A through 207H in the embodiment shown in FIG. 2A can be evening distributed circumferentially around the circumference along the transverse axis, such as transverse axis 407 shown in FIG. 4A which is perpendicular to the longitudinal axis of the delivery sheath 201. In another embodiment, the openings are not evenly distributed circumferentially. Each of the openings, such as opening 207A exposes the lumen within delivery sheath 201, which lumen is defined by the inner surface of the generally tubular wall, in one embodiment, of delivery sheath 201. The sheath 201 also includes an outer surface, and the opening forms a channel from the outer surface to inner surface of delivery sheath 201 in one embodiment. The implant 10 can be disposed within the distal end of delivery sheath 201 such that the distal portion of the implant, while the implant is being deployed, is exposed while the remainder of the implant 10 remains within the lumen of delivery sheath 201 until the delivery sheath 201 is retracted proximally. FIG. 8B shows an example of how the implant device 10 can be disposed within the lumen of a delivery sheath while only the very distal portion of the implant 10 extends distally beyond the distal opened end 205.

FIG. 2B shows another embodiment of a delivery sheath. The delivery sheath 211 shown in FIG. 2B has a plurality of openings, such as openings 214A through 214H, and these openings are disposed near the distal end 212 of delivery sheath 211. The distal end 212 includes the distal open end 214 which exposes the internal lumen of delivery sheath 211. The openings shown in FIG. 2B occupy only the distal portion and terminate at portion 213 which is proximal of the distal end 212. In the example shown in FIG. 2B, each of the openings is a closed form curve, which in this case can be a circle; in other embodiments an oval or ellipse or other closed formed curves can be used. In the example shown in FIG. 2B, the circles are of constant size; that is there is no variation in size in the different openings. There is no overlap between the openings and the density of the openings is consistent across the various regions; in other words, there is no variation across zones as in the case of FIG. 2C or 2D where the density of the openings varies between the zones. There is no solid band, presumably because there is no known kink point. In one embodiment, the outer coil 12 can be in direct contact with the openings and will slide against the openings, such as openings 214A through 214H when delivery sheath 211 is moved relative to implant 10 when implant 10 is being deployed. In one embodiment, the openings shown in FIG. 2B can be included near the distal end in a range of about ½ inch to 6 inches from the distal end such that they occupy about ½ inch to about 6 inches of the distal portion of delivery catheter 211 which can be, in one embodiment, over 40 centimeters long.

FIG. 2C shows another embodiment of a delivery sheath of the present invention. Delivery sheath 221 includes four distinct zones, zones 1 through zone 4; each of the zones have openings, and each opening is of the same size within a particular zone but the openings in different zones have different sizes. For example, openings 225 in zone 1 are larger than openings 226 in zone 2. Similarly, openings 226 in zone 2 are larger in surface area than openings 227 in zone 3. Further, openings 227 in zone 3 are each larger than the openings 228 in zone 4. Moreover, there is a variation in density in the openings between or among the zones. For example, there are fewer openings in zone 4 than in zone 3 on a per surface area basis. That is, the surface area within zone 4 includes fewer openings than the openings in that same surface area within zone 3. As shown in FIG. 2C, there is also a different amount of overlap between the openings. For example, there is no overlap between the openings 228 in zone 4 whereas there is considerable overlap between the openings 225 in zone 1, and there is less overlap in zone 2 among the openings 226 then the overlap among openings 225 in zone 1. The amount of overlap decreases from the distal end 224 up to the start region 223 which is near the portion 222 which is proximal of the distal end 224 of delivery sheath 221. The distal end 224 also includes a distal open end which is open to the lumen of the delivery sheath 221. As shown in FIG. 2C, the size and orientation and shape of each of the openings can be configured such that they will not snag or hang up the outer coil 12 as the sheath 221 is retracted proximally towards the handle to which it is connected. The openings shown in FIG. 2C can be diamond shaped openings, and the acute angles in these diamond shaped openings can be bisected by the longitudinal axis of delivery sheath 221 (see for example FIG. 4C). When the diamonds are cut into the delivery sheath 221 in a nested pattern as shown in FIG. 2C, the material that remains provides a basket weave looking structure, and that material that remains between the diamonds can be referred to as a strut. The removal of material in the form of the diamond cut holes provides flexibility in the tubing, which can be a polyimide material or other materials known in the art which are appropriate for sheaths or other tubings in catheter systems. The improved flexibility in the tubing facilitates canulation of torturous pathways in the body, such as the uterine cavity and the fallopian tubes. The struts which remain in the material after the diamond openings are cut or otherwise formed, provides strength in the radial direction of the delivery sheath or other tube, which aides in keeping the lumen of the delivery sheath open while it is being deflected; it also helps with preventing the formation of kinks in the delivery sheath or catheter system during the deflection. The diamond size and shape combination results in a condition where the diamonds when packed tightly enough in a particular section which still provide enough material left to form the struts and provide enough radial strength in that section but while also giving desired flexibility. The example shown in FIG. 2C is one in which the pattern of openings or diamond cuts is one where the openings are more closely packed in the most distal zone and they progressively get more sparsely packed in subsequent zones as they move proximally along the catheter towards portion 222 which is proximal of the distal end 224. The resulting flexibility profile is one in which the delivery sheath is most flexible at the distal end and gradually gets stiffer towards the proximal end near zone 4. The start point 223 can be, in one embodiment, as far away from distal end 224 as two to three inches or six inches or can be as close to the distal end as about one inch.

FIG. 2D shows another embodiment of a delivery sheath according to the present invention. Delivery sheath 231 in FIG. 2D includes three zones, each having openings of the same size (surface area) but there is a variation in density among the three zones such that zone 1 has the highest density of openings while zone 2 has the second highest density of openings within its surface area and lastly zone 3 has the lowest density of openings within this surface area. The openings 234 as shown in FIG. 4D can be diamond shaped with acute angles for two of the four angles or can be squares or can be quadralaterals such as rectangles or triangles or polygons having more than four sides. It will be appreciated that in the various other embodiments described herein, the openings can have these various different geometries as well as alternatively having closed form curves, such as circles, ovals, ellipses, etc. As shown in FIG. 2D, the three zones occupy a relatively small portion beginning near portion 232 which is proximal of the distal end 233 of the delivery sheath 231 which can be over 12 inches long and in one embodiment over 40 centimeters long. FIG. 2D also shows that the struts 235, 236, and 237 have different sizes in the three different zones as indicated in FIG. 2D.

It will be understood that another alternative embodiment of a delivery sheath can include one in which the openings vary in size but that there is a constant density among different zones, where each zone is defined by one size of the openings. In one embodiment, the variation in size places the larger openings in the distal or near the distal end and the smaller openings are proximal of the distal end.

FIGS. 3A, 3B, 3C, 3D, 3E, and 3F depict an embodiment of a delivery sheath according to the present invention. Delivery sheath 301 in this embodiment includes three zones, zone 1, zone 2, and zone 3, each of which contain openings that have the same size, which are labeled as opening 303. The openings 303 are more densely packed in zone 1 than the openings in zone 2, and similarly, the openings 303 in zone 2 are more densely packed than the openings 303 in zone 3. Zone 3 ends near the portion 302 which is proximal of the distal end 304 which is adjacent to the distal open end which opens into the lumen of the delivery sheath 301. In one embodiment, the very distal portion of implant device 10 can extend beyond the distal open end 304 such as in the example shown in FIG. 8B during deployment of the implant device 10. FIG. 3B shows examples of sizes, in inches, within a particular portion of zone 1. The size labeled with the numeral 3 in a box applies to all three zones in FIGS. 3A, 3C, and 3E. FIG. 3D shows a detailed view of a portion of zone 2, and FIG. 3E shows a detailed view of the portion of zone 3. It will be appreciated that the various measurements, shown in inches, are merely an example of one embodiment of delivery sheath 301 and alternative sizes can be used in alternative embodiments.

FIGS. 4A, 4B, 4C, 4D, and 4E show various examples of openings which can be used in any one of the embodiments described herein. Moreover, these figures also show one example of an orientation of each of the openings relative to an axis of a delivery sheath in order to optimize deployment of an implant or other device from the delivery sheath or to otherwise allow the delivery sheath to provide or perform an operation as described herein. For example FIG. 4A shows openings 404 and 405 which are shaped as squares and which are oriented relative to longitudinal axis 403 such that no edge of openings 404 and 405 is perpendicular to the longitudinal axis 403. The longitudinal axis 403 can be the axis down the center of the lumen formed by delivery sheath 401 from the proximal portion of delivery sheath to the distal end such as the distal open end 402. A transversal or radial axis 407 is perpendicular to the longitudinal axis 403, and it can be seen that no edge of the openings 404 or 405 is perpendicular to longitudinal axis 403. In one embodiment, two of the angles formed by the sides of the openings 404 and 405 are bisected by a transversal or radial axis while two of the other angles formed by the sides of the openings are bisected by longitudinal axis 403. In this manner, the orientation of the openings minimizes the chances of an implant device, such as the outer surface of outer coil 12 catching or snagging on an edge of the openings. FIG. 4B shows a more detailed view of the opening 405 and its orientation relative to the longitudinal axis 403. It can be seen that opening 405, like opening 404, is a square having sides which are less than 0.02 inches long. The sizes shown in FIGS. 4B, 4C, 4D and 4E are in inches. The opening 410 shown in FIG. 4C is a diamond shaped opening with two acute angles opposing each other and bisected by a longitudinal axis 403A (or the line which bisects those angles is parallel with the longitudinal axis 403A). Thus the shape and orientation of the diamond shaped opening 410 is such that no edge of the diamond shaped opening 410 is perpendicular to the longitudinal axis, and this minimizes the likelihood that the outer surface of the outer coil 12 will catch or snag on one of the edges of an opening during deployment of implant device 10, such as when the sheath having the opening 410 is retracted proximally toward the handle in one embodiment. FIG. 4D shows an example of a diamond like shape in which the edges are rounded to give the opening 433 as shown in FIG. 4D. Two of the angles are again bisected by the longitudinal axis 403B which is parallel with the axis down the length from the proximal end to the distal end of a particular delivery sheath, such as longitudinal axis 403 shown in FIG. 4A. FIG. 4E shows an example of an oval shaped opening 437. This oval is oriented such that the two smaller sides of the oval are bisected by the longitudinal axis 403 in order to minimize any curve which presents an edge that is parallel with the transverse or radial axis, such as transverse or radial axis 407 of a delivery catheter or delivery sheath.

FIGS. 5A and 5B show cross sectional views, taken at the lines labeled 5A shown in FIG. 3A. In particular, FIG. 5A shows the delivery sheath 301 in cross sectional views and shows the openings 303 circumferentially around the circumference of the delivery sheath 301. The center point 501 is along the transverse or radial axis 407A which is perpendicular to the longitudinal axis of the delivery sheath 301. The material between the openings 303 shown in FIG. 5A and in FIG. 5B represent the struts described herein which provide the strength in the radial direction of the delivery sheath 301. While FIG. 5A shows the delivery sheath 301 without a device disposed within its lumen, FIG. 5B shows a cross sectional view of delivery sheath 301 with a cross sectional view of a device, such as the implant device 10 disposed within the lumen of delivery sheath 301. In particular, a portion of outer coil 12 can be seen in the cross sectional view of FIG. 5B along with a portion of the inner coil 18 which coaxially surrounds inner wire 17 as shown in FIG. 5B. In one embodiment, the inner wire 17 can extend from the distal ball 20 to the proximal end of inner coil 18. It can be seen from FIG. 5B that the outer surface of the outer coil directly contacts and abuts the inner surface of the delivery sheath 301 such that when the delivery sheath is moved relative to the implant device 10, the outer surface of the outer coil 12 will slide against the openings 303. In one embodiment, the delivery sheath, such as delivery sheath 303 can have a constant and consistent wall thickness from its proximal end (such as the end at which it is attached to the handle which controls the deployment of an implant device) all the way to its distal end. Having a constant or consistent wall thickness simplifies manufacturing of the delivery sheath. The wall thickness can be selected such that the proximal portion of the delivery sheath provides sufficient pushability and sufficient trackability can be provided at the distal end by created or forming openings at the distal end as described herein. The consist or constant wall thickness is shown as distance or thickness 503 in FIG. 5A in one embodiment, and this thickness is defined by the distance between the inner surface of the delivery sheath and the outer surface of the delivery sheath. In one embodiment, the proper selection of the material used to form the delivery sheath and the thickness of this wall can allow for a sheath having a consistent or constant wall thickness through its entire length which can be stiff enough at the proximal end but which can still be less thick than the wall of a catheter that is reinforced with coils or braids or other mechanisms.

In one embodiment, the delivery sheath can include a solid band or section, along the longitudinal length of the delivery sheath, that interrupts or separates one set of openings from another set of openings. The solid band or section can be positioned, along the longitudinal length of the delivery sheath, at a predetermined point that tends to kink, if the solid band or section is not present, when the delivery sheath is used, in a typical physiological setting, to deploy one or more devices or to otherwise perform one or more operations. For example, the predetermined point can be about 10 to about 25 millimeters from the distal end of the delivery sheath.

FIG. 6 shows an example of a delivery sheath which includes such a solid band or section. The solid band 607 is disposed within zone 4 or zone 606 in the delivery sheath 601. The delivery sheath 601 includes four zones 603, 604, 605, and 606. In this embodiment, each zone has openings of the same size within a zone, but the openings in different zones are different sizes. For example, each opening 610 in zone 603 is larger than each opening 611 in zone 604. Similarly, opening 611 is larger than opening 612 in zone 605. Similarly, openings 613 in zone 606 are smaller than openings 612 in zone 605. Hence, the largest openings are at the distal end 602 and the smallest openings are near the portion 614 which is proximal of the distal end 602. A delivery sheath 601 can be tested without the solid band to determine if it has the tendency to kink at certain points and a solid band or section can be introduced at those points after the testing. The patterns of openings can be customized in a way that serves the flexibility/stiffness requirement in an optimal way. It can be configured in a way that supports the structure or body that it is covering in a complimentary way. For instance, if there is a section of the inner structure that is too flexible or too stiff, the openings can be arranged in a pattern with various degrees of flexibility or stiffness that is the opposite of the inner structure's flexibility or stiffness to increase support to sections that are too flexible or to promote flexibility to sections that are too stiff. For example, in a particular catheter and implant subassembly, there can be a section that is too flexible which is then followed immediately by a stiff section. This phenomenon can create a kink point in the system. The sheath can then be designed to have a pattern of openings that has a gap in the pattern at the same location to create a stiff section to support the flexible kink spot and make it stiffer, which should eliminate the tendency to kink at that location. The same concept can be applied with sections that are too stiff where the diamond pattern can be designed to be very flexible and provide or promote flexibility at the stiff section.

FIGS. 7A and 7B show one example of an embodiment which can include a sleeve which is disposed over the outer surface of the delivery sheath in one or more regions containing the openings. In one embodiment, the material of the sleeve can be different than the material of the sheath; for example, the sleeve can be formed from a material which is considerably more flexible than the material forming the delivery sheath in order to prevent the sleeve from reducing the flexibility imparted into the delivery sheath by the formation of the openings in the delivery sheath. This sleeve can also or alternatively have a very thin wall thickness in order to not reduce the flexibility of the region containing the openings. This sleeve can at least partially restrict the flow of fluid, such as a physiological fluid or a distention fluid or an imaging fluid, etc. through the openings into the lumen of the delivery sheath. For example, if the implant device, such as the implant device 10 includes a hydrogel component, it can be desirable to prevent the flow of fluid into the lumen during deployment to thereby prevent the hydrogel from swelling when it is exposed to the fluid. Delivery sheath 701 shown in FIG. 7A includes such a sleeve 704 which restricts the flow of fluids into the lumen of delivery sheath 701 through the openings 705. The sleeve 704 can be applied over all the openings in a region of the delivery sheath 701 such that it blocks the flow of fluid into all of the openings of that region. The distal open end 702 can also be occluded by placing a material (described below) into the open distal end or providing a cap or some other mechanism to seal the open distal end. FIG. 7B shows a cross sectional view, taken at line 7B-7B as shown in FIG. 7A. It can be seen from the cross sectional view that the sleeve 704 closely abuts the outer surface of the delivery sheath 701 and thereby blocks or restricts the flow of fluid into the opening 705 when the sleeve is applied over the delivery sheath 701 in the region of the openings 705. When the sleeve is formed from a composition of material that is different in the composition forming the delivery sheath, the sleeve can be constructed in a manner that it does not restrict the added flexibility created by the opening 705. For example, if the composition forming the sleeve 704 is much more flexible than at least the proximal portion of the sheath around the region containing the opening 705, then the sleeve 704 does not impact the increased flexibility or retractability of the distal portions of the delivery sheath while at the same time retaining the ability to restrict the flow of fluids into the lumen of the delivery sheath by blocking the opening 705.

As noted with respect to FIG. 7A, the distal open end of any one of the delivery sheaths described herein can also include a material that is placed on or into the distal open end in order to restrict the flow of fluid into the distal open end of the lumen of the delivery sheath. For example, this material can be a jelly, such as a petroleum jelly that is stuffed into the open distal end of the lumen of the delivery sheath. For example, petroleum jelly or some other jelly or hydrophobic material or hydrophilic material can be stuffed into the distal open end 702.

FIG. 8A shows an example of a delivery catheter 801 which includes a sleeve 804, which is similar to the sleeve 704, and which covers openings 805 in the delivery catheter 801. A material 802 has been stuffed into the open distal end of the delivery catheter 801, and this material can block the flow of fluid into the lumen and down into the delivery catheter 801 past the portion 803 which is proximal of the distal end of the delivery catheter 801. In one embodiment, the material can be one of a pierceable hydrophobic or hydrophilic material or can be a pierceable seal or cap that is attached to the distal end or it can be a dissolvable seal or cap that attaches to the distal end. The material 802 as well as the sleeve 804 can serve to restrict the flow of fluids into the lumen of the delivery catheter 801, and this can be particularly useful when the implant device in the delivery catheter contains a hydrogel component or other component which needs to be protected against fluids during deployment. In one embodiment, a distal end of the implant device itself can extend out beyond the distal end of the delivery catheter through the material 802 while at the same time the material at least partially restricts the flow of fluid into the distal end. This is shown in FIG. 8B in which a small portion of the input device 10 extends beyond the material 802. In the example shown in FIG. 8, only the distal ball 20 and a portion of the inner coil 18 which is distal of the hydrogel components 22 extends beyond the material 802 which can still restrict the flow of fluid even after the distal ball and the small portion of the inner coil 18 extend beyond the material 802.

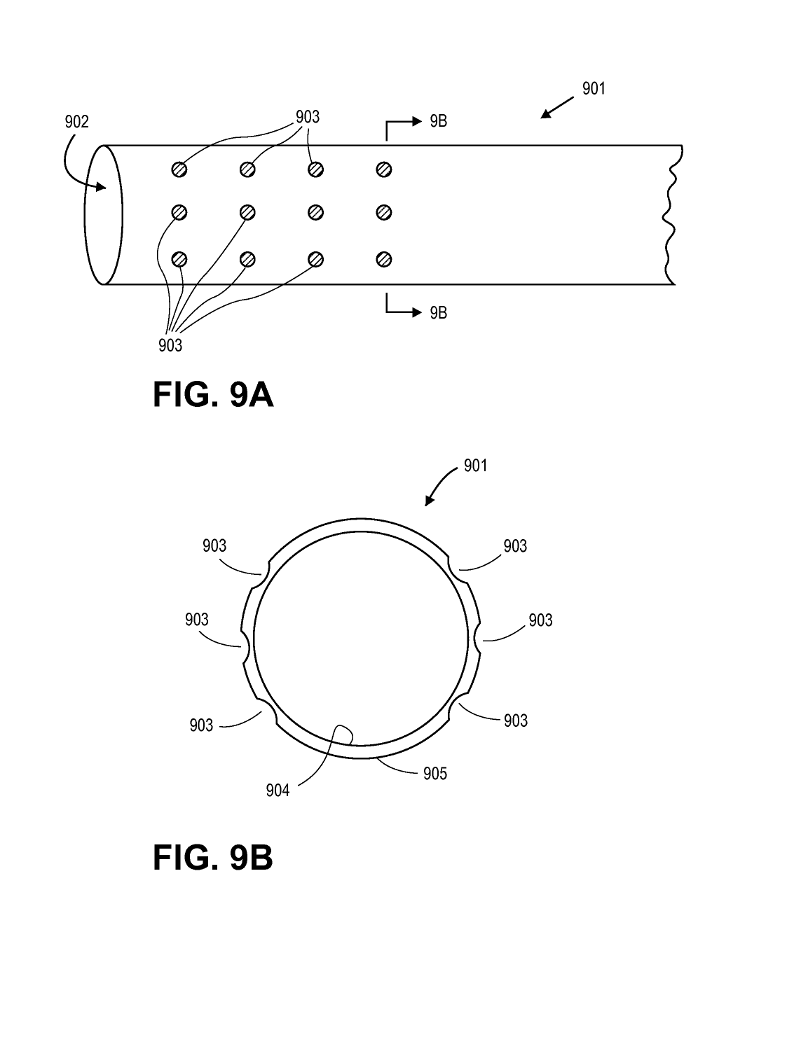

FIGS. 9A and 9B show an example of another embodiment of a sheath or catheter component that uses dimples, instead of openings or in addition to openings. The dimples can be depressions (resembling craters) in one or more surfaces (e.g., inner and/or outer surfaces) of the delivery sheath. The dimples can, like the openings described herein, provide a variation in flexibility in the portion of the sheath that includes the dimples relative to other portions of the sheath that do not include the dimples. The portion that includes the dimples can be near a distal end of the sheath. The dimples, in one embodiment, are separate and distinct from each other and can have a shape selected from one of (a) a closed form curve such as a circle or oval or ellipse; (b) a triangle; (c) a polygon having four or more sides. The dimples can be arranged in patterns described herein, such as the patterns shown in FIG. 2A-2D, 3A, 6, or 7A; for example, the dimples can be arranged in zones or regions that have dimples of different sizes or different densities or both, etc. The dimples can be larger and more dense near the distal end and smaller and less dense proximal of the distal end; alternatively, the dimples can have the same size across the zones but be more densely packed in a distal zone than the dimples in a zone that is proximal of the distal zone.

The dimples 903, shown in FIGS. 9A and 9B, are near the distal open end 902 of sheath 901 and are circular dimples that resemble a crater or depression in which there is less material in the sheath's wall than surrounding regions of delivery sheath 901. This can be seen in the cross-section view of FIG. 9B which shows a cross section of delivery sheath 901 (taken at line 9B-9B in FIG. 9A). The dimples 903, as shown in FIG. 9B, extend into only a portion of the wall thickness of the sheath 901; the dimples 903 are not through holes that go completely through the wall of sheath 901. The wall thickness of sheath 901, as shown in FIG. 9B, is defined by the distance between the outer surface 905 of sheath 901 and inner surface 904 of sheath 901. The inner surface 904 defines the lumen of the sheath 901 and no dimple punctures that inner surface 904 in the embodiment shown in FIG. 9B. While FIG. 9B shows an example of dimples that have a curved, crater-like depression, it will be appreciated that the dimples can have straight internal edges (that resemble a box). The dimples reduce the wall thickness of sheath 901 in those areas of the sheath 901 that are occupied by the dimples.

The delivery sheath described herein can be formed from a variety of materials, including for example, polyimide, provided in either a thermoset or thermoplastic form. For example, thermoset polyimide can be molded in a cylindrical form having a wall thickness of many thousandths of an inch to less than one thousandth of an inch, while maintaining favorable axial stiffness. However, alternative materials may be selected depending on the conditions of use, e.g. the resilience and flexibility that is required of the delivery sheath described herein. For example, in various embodiments, suitable alternatives to polyimide may include polyamides, polyurethanes, fluoropolymers, or polyetheretherketone (PEEK).

The openings can be formed using techniques which are known in the art depending upon the materials used to form the delivery sheath. For example, the openings can be formed by a laser which cuts through the material; the laser can be computer controlled to quickly generate the openings. The catheter can be placed on a mandrel or other structure to hold it in place while the laser cuts the openings. In other embodiments, the openings can be molded into the delivery sheath or drilled into the delivery sheath with a mechanical drill or mechanical saw. In other embodiments, the openings can be etched, either chemically or physically (such as through a mask) into the delivery sheath using techniques that are known in the art.

The dimples described herein can be formed using techniques which are known in the art depending upon the materials used to form the delivery sheath. For example, the dimples can be etched, either chemically or physically (such as through a mask), into the delivery sheath using techniques that are known in the art. The dimples can also be formed with a laser or a mechanical drill or saw or other mechanism. The dimples can also be formed in a molding process which forms or creates the delivery sheath.

While this description has emphasized the use of these openings near the distal end of a delivery sheath or other tubing used in a medical operation, it will be appreciated that in alternative embodiments, it may be appropriate to place the openings in a middle portion or some other portion of a medical tubing or delivery sheath.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

1. A catheter comprising:
an expandable implant having an outer surface;
a handle coupled to the expandable implant;
a delivery sheath coupled to the handle, the delivery sheath having an outer surface and a lumen in which the expandable implant is disposed, the lumen defined by an inner surface of the delivery sheath and the delivery sheath having a plurality of openings near a distal end of the delivery sheath, the openings extending from the inner surface to the outer surface of the delivery sheath.

2. The catheter as in claim 1 wherein the openings are in direct physical contact with the outer surface of the expandable implant and wherein the openings are configured in at least one of size, shape and orientation to allow the outer surface of the expandable implant to slide along the openings.

3. The catheter as in claim 2 wherein the expandable implant comprises a first coil which forms the outer surface of the expandable implant and the expandable implant comprises a second coil that is coupled to the first coil and is coaxially surrounded by the first coil and the expandable implant comprises a hydrogel coupled to the second coil.

4. The catheter as in claim 2 wherein the distal end of the delivery sheath is sized to allow the expandable implant to be deployed through the distal end and wherein the expandable implant is configured to slide along the openings as the expandable implant is deployed.

5. The catheter as in claim 4 wherein the plurality of openings are configured to make the delivery sheath more flexible in the region containing the plurality of openings than a region of the delivery sheath which does not contain openings.

6. The catheter as in claim 5 further comprising:
A sleeve disposed over the outer surface of the delivery sheath in the region containing the plurality of openings.

7. The catheter as in claim 6 wherein the expandable implant comprises a hydrogel and wherein the sleeve restricts the flow of fluid into the lumen to reduce any swelling of the hydrogel while the hydrogel is in the lumen and wherein the distal end of the delivery sheath includes a material that restricts the flow of fluid into the distal end.

8. The catheter as in claim 7 wherein the material is one of: (a) pierceable hydrophobic or hydrophilic material or (b) pierceable seal or cap that attaches to the distal end or (c) a dissolvable seal or cap that attaches to the distal end.

9. The catheter as in claim 7 wherein, during deployment of the expandable implant a distal end of the expandable implant extends out beyond the distal end of the delivery sheath while the material restricts the flow of fluid into the distal end.

10. The catheter as in claim 5 wherein the delivery sheath includes a solid band which separates a first set of opening in the plurality of openings from a second set of openings in the plurality of openings, and wherein the solid band is positioned, along an longitudinal length of the delivery sheath, at a predetermined point that tends to kind when the delivery sheath is used to deploy the expandable implant if the solid band is not present.

11. The catheter as in claim 10 wherein the predetermined point is about 10 to about 25 mm from the distal end of the delivery sheath.

12. The catheter as in claim 5 wherein the openings have a shape selected from a triangle, a quadrilateral or a closed form curve.

13. The catheter as in claim 5 wherein the openings vary in size such that openings near the distal end are larger than openings that are proximal of the distal end.

14. The catheter as in claim 5 wherein the sheath has different regions of openings, the different regions including a first region and a second region, the first region having a higher density of openings than the second region.

15. The catheter as in claim 13 wherein the sheath has different regions of openings, the different regions including a first region and a second region, the first region having a higher density of openings than the second region and wherein openings in the first region are each larger than the openings in the second region.

16. The catheter as in claim 5 wherein the plurality of openings occur in a region on the delivery sheath that is about ½ inch to about 6 inches long and wherein each of the openings has at least two sides or is a closed form curve.

17. The catheter as in claim 16 wherein a side of each of the openings is less than 0.05 inches.

18. The catheter as in claim 17 wherein each side of each of the openings is less than 0.05 inches.

19. The catheter as in claim 18 wherein the sheath has different regions of openings, the different regions including a first region and a second region, the first region having a higher density of openings than the second region, the first region being distal of the second region.

20. The catheter as in claim 19 wherein no side of each of the openings is perpendicular to a longitudinal axis of the delivery sheath, the longitudinal axis extending from the distal end to a proximal end of the delivery sheath.

21. The catheter as in claim 20 wherein the delivery sheath is formed from a polyimide tubing and the openings are cut or etched into the polyimide tubing.

22. The catheter as in claim 5 wherein the delivery sheath has a constant wall thickness from its proximal end to the distal end.

23. The catheter as in claim 5 wherein the delivery sheath has a varying wall thickness.

24. The catheter as in claim 5 wherein the delivery sheath has a varying diameter.

25. A catheter comprising:
a handle;
a delivery sheath coupled to the handle at a proximal end of the delivery sheath, the handle configured to control the delivery sheath, the delivery sheath having a consistent wall thickness from its proximal end to its distal end and having a lumen configured to perform a medical operation, the lumen defined by an inner surface of the delivery sheath which has an outer surface, the consistent wall thickness being defined by a distance between the inner surface of the delivery sheath and the outer surface of the delivery sheath, the delivery sheath having a plurality of openings near the distal end of the delivery sheath, each of the openings being separate and distinct from the other openings in the plurality of openings and each of the openings extending from the inner surface to the outer surface and each of the openings having a non-negligible surface area.

26. The catheter as in claim 25 wherein the openings are configured in at least one of size, shape and orientation to allow an outer surface of a medical device, which is used in the medical operation, to slide along the openings and wherein the proximal end of the delivery sheath is stiffer than the distal end having the plurality of openings.

27. The catheter as in claim 25 wherein the handle is configured to retract the delivery sheath proximally toward the handle.

28. A catheter comprising:
a handle;
a delivery sheath coupled to the handle at a proximal portion of the delivery sheath, the handle configured to control the delivery sheath, the delivery sheath having a plurality of dimples in a first portion of the delivery sheath, the plurality of dimples providing a first flexibility in the first portion, the first flexibility being different than the flexibility in another portion of the delivery sheath.

29. The catheter as in claim 28 wherein the plurality of dimples are each separate and distinct from each other and each of the dimples have a shape selected from one of (a) a closed form curve; (b) a triangle or (c) a polygon having four or more sides.

30. The catheter as in claim 29 wherein the delivery sheath has different densities of the dimples in different regions of the first portion.

31. The catheter as in claim 29 wherein the delivery sheath has different sizes of the dimples in different regions of the first portion.

32. The catheter as in claim 30 wherein the first portion is near a distal end of the delivery sheath.

33. An expandable implant deployable by a catheter system comprising:
a first coil having a distal end and a proximal portion;
a second coil coupled to the first coil at the proximal portion;
a first hydrogel component having a first face, the first hydrogel component coupled to the first coil; and
a second hydrogel component coupled to the first coil and having a second face which is coupled apposed to the first face.

34. The expandable implant of claim 33 wherein a portion of the first coil is coaxially surrounded by the second coil.

35. The expandable implant of claim 34 wherein the first coil is coaxially surrounded by the second coil such that at least a portion of the first coil is not coaxially surrounded by the second coil at the distal end.

36. The expandable implant of claim 34 wherein the first coil is coaxially surrounded by the second coil such that at least a portion of the second coil does not coaxially surround the first coil at the proximal portion.

37. The expandable implant of claim 33 wherein the portion of the first coil between the first and second hydrogel components is stretched out.

38. The expandable implant of claim 33 wherein the first and second hydrogel components are cone-shaped.

39. The expandable implant of claim 38 wherein the first and second faces are the smaller ends of the first and second hydrogel components respectively.

40. The expandable implant of claim 39 further comprising:
an atraumatic distal ball coupled to the first coil at the distal end.
